(19) United States
(12) Patent Application Publication
(10) Pub. No.: US 2019/0184143 A1
(43) Pub. Date: Jun. 20, 2019

(54) MEDICAL DEVICE FOR ACCESSING AND/OR TREATING THE NEURAL VASCULATURE

(71) Applicant: BOSTON SCIENTIFIC SCIMED, INC., MAPLE GROVE, MN (US)

(72) Inventors: DAVID JOHN ONUSHKO, MAPLE GROVE, MN (US); JOEL MUNSINGER, BLAINE, MN (US); BRIAN R. REYNOLDS, RAMSEY, MN (US); JOSE A. MEREGOTTE, BLAINE, MN (US)

(73) Assignee: BOSTON SCIENTIFIC SCIMED, INC., MAPLE GROVE, MN (US)

(21) Appl. No.: 16/219,924

(22) Filed: Dec. 13, 2018

Related U.S. Application Data
(60) Provisional application No. 62/599,416, filed on Dec. 15, 2017.

(51) Int. Cl. A61M 25/10; A61M 25/09

(52) U.S. Cl. CPC A61M 25/104; A61M 25/1002; A61M 2025/09183; A61M 2025/09008; A61B 2217/005; A61M 25/09

(57) ABSTRACT

Medical devices and methods for making and using a medical device are disclosed. An example medical device may include a guidewire for accessing and treating a region of the neural vasculature. The guidewire may include an elongate shaft having a distal end region. The elongate shaft may include an inner tubular member and an outer tubular member. The inner tubular member may have a plurality of openings formed therein. The outer tubular member may have a plurality of slots formed therein. An occlusive balloon may be coupled to the distal end region of the elongate shaft and disposed about at least some of the plurality of openings. The inner tubular member may define a lumen in fluid communication with the occlusive balloon through the plurality of openings. The tip member may be coupled to the distal end region of the elongate shaft.

### CROSS-REFERENCE TO RELATED APPLICATIONSs

This application claims the benefit of priority under 35 U.S.C. § 119 of U.S. Provisional Application No. 62/599,416, filed Dec. 15, 2017, the entire disclosure of which is hereby incorporated by reference.

### TECHNICAL FIELD

The present disclosure pertains to medical devices, and methods for manufacturing medical devices. More particularly, the present disclosure pertains to medical devices for accessing and/or treating the neural vasculature.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An method for treating a vascular region is disclosed. The method comprises: advancing a guidewire through a neural blood vessel to a first position adjacent to an embolism, the guidewire comprising: an elongate shaft having a distal end region, the elongate shaft including an inner member and an outer member, wherein the inner member has a plurality of openings formed therein, and an inflatable balloon coupled to the distal end region; advancing the guidewire to a second position distal of the embolism; inflating the inflatable balloon; advancing a treatment catheter over the guidewire to a position adjacent to the embolism; and aspirating the embolism.

Alternatively or additionally to any of the embodiments above, at least a portion of the guidewire has an outer diameter of 0.01 to 0.018 inches.

Alternatively or additionally to any of the embodiments above, the inner member includes a tubular member defining a lumen.

Alternatively or additionally to any of the embodiments above, the lumen is an inflation lumen, wherein the inflatable balloon is positioned about at least some of the plurality of openings, and wherein inflating the inflatable balloon includes passing inflation media through the inflation lumen and at least some of the plurality of openings.

Alternatively or additionally to any of the embodiments above, the outer member includes a polymeric sleeve.

Alternatively or additionally to any of the embodiments above, the outer member includes a tubular member with a region having a plurality of slots formed therein.

A guidewire for accessing and treating a region of the neural vasculature is disclosed. The guidewire comprises: an elongate shaft having a distal end region, the elongate shaft including an inner tubular member and an outer tubular member; wherein the inner tubular member has a plurality of openings formed therein; wherein the outer tubular member has a plurality of slots formed therein; an occlusive balloon coupled to the distal end region of the elongate shaft and disposed about at least some of the plurality of openings; wherein the inner tubular member defines a lumen in fluid communication with the occlusive balloon through the plurality of openings; and a tip member coupled to the distal end region of the elongate shaft.

Alternatively or additionally to any of the embodiments above, the plurality of slots include a first plurality of slots disposed along a first region of the outer tubular member, a second plurality of slots disposed along a second region of the outer tubular member, and a third region positioned between the first region and the second region that is free of slots.

Alternatively or additionally to any of the embodiments above, the occlusive balloon includes a proximal waist and wherein the proximal waist is coupled to the second region.

Alternatively or additionally to any of the embodiments above, the outer tubular member includes a fourth region disposed adjacent to the third region, the fourth region being free of slots.

Alternatively or additionally to any of the embodiments above, the occlusive balloon includes a distal waist and wherein the distal waist is coupled to the fourth region.

Alternatively or additionally to any of the embodiments above, further comprising a sleeve disposed between the distal waist and the fourth region of the outer tubular member.

Alternatively or additionally to any of the embodiments above, at least some of the plurality of openings in the inner tubular member are circular in shape.

Alternatively or additionally to any of the embodiments above, at least some of the plurality of openings in the inner tubular member are non-circular in shape.

Alternatively or additionally to any of the embodiments above, the tip member has a closed distal end.

Alternatively or additionally to any of the embodiments above, the tip member includes a polymer jacket.

Alternatively or additionally to any of the embodiments above, the tip member includes a coil.

Alternatively or additionally to any of the embodiments above, the outer tubular member includes a distal portion, a proximal portion, and a connector coupling the distal portion to the proximal portion.

A guidewire for accessing and treating a relatively small vasculature region is disclosed. The guidewire comprises: an inner tubular member having a distal region with a plurality of openings formed therein; an outer tubular member disposed about the inner tubular member, the outer tubular member having a first region having a first plurality of slots formed therein, a second region having a second plurality of slots formed therein, and a third region disposed between the first region and the second region, the third region being free of slots; a balloon coupled to the third region of the outer tubular member; wherein the inner tubular member defines a lumen in fluid communication with the balloon through the plurality of openings; and a closed tip member extending distally from a distal end region of the outer tubular member.

Alternatively or additionally to any of the embodiments above, the outer tubular member includes a fourth region free of slots and wherein a sleeve is disposed along the fourth region and positioned between the outer tubular member and the balloon.

Alternatively or additionally to any of the embodiments above, a jacket is disposed along at least a portion of the outer tubular member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional side view of an example medical device.

FIG. 2 is a partial cross-sectional side view of an example medical device.

FIG. 3 is a partial cross-sectional side view of an example medical device.

FIG. 4 is a partial cross-sectional side view of an example medical device.

FIGS. 5-9 schematically illustrate an example process for using a medical device.

FIG. 10 is a side view of an example accessory device for use with a medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Available options for treating stroke due to thromboembolism are limited. For example, it may be difficult to reach occlusions, embolisms, or the like in regions of the vasculature such as regions of the neural vasculature. Disclosed herein are devices that can be navigated to a region adjacent to an occlusion, embolism, or the like in order to allow for treatment/removal of the embolism. The devices may allow for removal of the embolism while minimizing the release of emboli distally. Some additional features of a number of devices are disclosed herein.

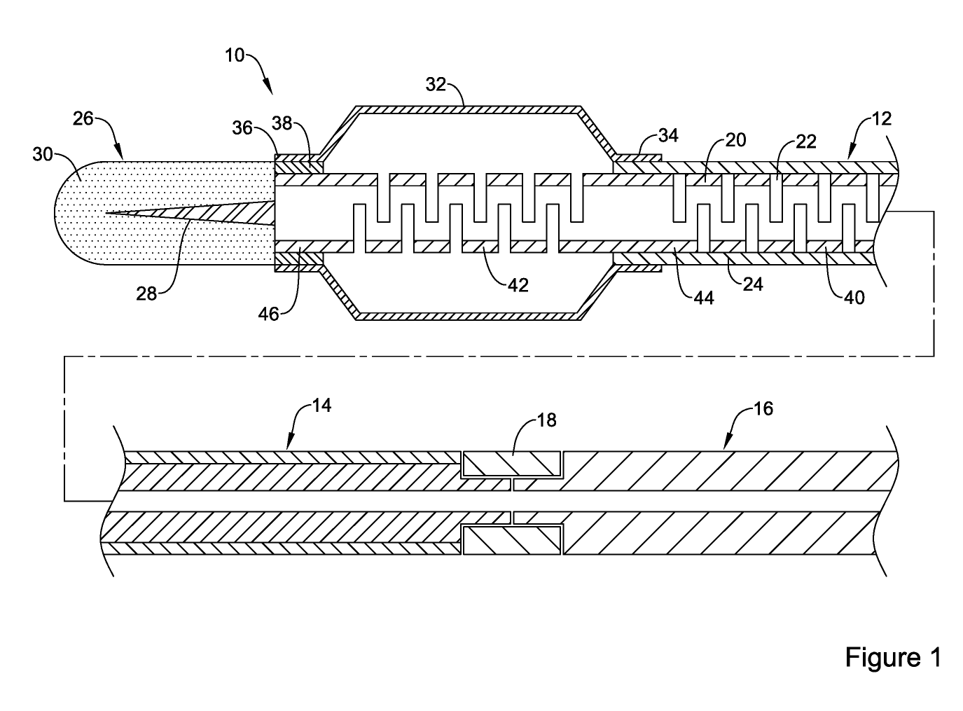

FIG. 1 illustrates an example medical device 10. In this example, the medical device 10 takes the form of a guidewire. The guidewire 10 may be designed to access a variety of locations within the anatomy. For example, the guidewire 10 may be designed to access portions of the neural vasculature. In addition or in the alternative, the guidewire 10 may be designed to access a number of additional vascular locations including deeper blood vessels such as those below-the-knee. In at least some instances, the guidewire 10 may have an outer diameter in the range of about 0.01 to 0.02 inches, or about 0.01 to 0.015 inches, or about 0.014 inches. Such sizes may be suited for accessing blood vessel in the brain and in other locations. Alternatively, the guidewire 10 may have a larger outer diameter and be suited for other indications.

The guidewire 10 may include an elongate shaft 12 having a distal portion 14 and a proximal portion 16. In some instances, the distal portion 14 and the proximal portion 16 form a singular, common shaft formed from the same material(s). In other instances, the distal portion 14 and the proximal portion 16 may be separate components that are joined, attached, or otherwise coupled to one another. For example, the distal portion 14 and the proximal portion 16 may be coupled to one another by a connector 18. For example, a section of the outer surface of the portions 14/16 may be removed and the connector 18 may be disposed over the removed sections to join the portions 14/16 together. Alternatively, the connector may be simply disposed over the portions 14/16. Other bonds may also be used including welds, thermal bonds, adhesive bonds, or the like. If utilized, the connector 18 includes a material that desirably bonds with both the distal portion 14 and the proximal portion 16. For example, the connector 18 may include a nickel-chromium-iron alloy (e.g., INCONEL).

The shaft 12 may include an inner member 20. In at least some instances, the inner member 20 may take the form of a core wire (e.g., having a solid cross-section). Alternatively, the inner member 20 may take the form of a tube having a lumen defined therein. As explained in more detail herein, the lumen may be an inflation lumen. A plurality of slots 22 may be formed in the inner member 20. In at least some embodiments, the slots 22 are disposed along the distal portion 14 of the shaft 12 (e.g., the distal portion of the inner member 20). In at least some embodiments, the proximal portion 16 of the shaft 12 (e.g., the proximal portion of the inner member 20) lacks slots 22. However, the proximal portion 16 may include slots 22. The slots 22 may be desirable for a number of reasons. For example, the slots 22 may provide a desirable level of flexibility to the shaft 12 and/or the inner member 20 while also allowing suitable transmission of torque. The slots 22 may be arranged/distributed in a suitable manner. For example, the slots 22 may be arranged as opposing pairs of slots 22 that are distributed along the length of the distal portion 14 of the shaft (e.g., the distal portion of the inner member 20). In some embodiments, adjacent pairs of the slots 22 may have a substantially constant spacing relative to one another. Alternatively, the spacing between adjacent pairs may vary. For example, more distal regions of inner member may have a decreased spacing (and/or increased slot density), which may provide increased flexibility. In other embodiments, more distal regions of the inner member 20 may have an increased spacing (and/or decreased slot density). These are just examples. Other arrangements are contemplated.

An outer member 24 may be disposed along and/or about the inner member 20. In at least some instances, the outer member 24 may take the form of a coating, sleeve, or jacket that extends along at least a portion of inner member 20. The outer member 24 may be disposed along the inner member 20 in a manner that prevents fluid from passing from within the lumen of the inner member 20 through the slots 22 (e.g., along slotted regions of the inner member 20 where the outer member 24 is present). In some instances, the outer member 24 may be disposed along the full length of the inner member 20. In other instances, the outer member 24 may be disposed along one or more discrete regions of the inner member 20.

A tip member 26 may be coupled to the shaft 12 and extend distally therefrom. The shape, form, and/or configuration of the tip member 26 may vary. For example, in some instances, the tip member 26 takes the form of a polymeric or “poly” tip. For example, the tip member 26 may include a shaping member 28 and a jacket 30 disposed above the shaping member 28. Other tip members 26 are contemplated. In at least some instances, the tip member 26 is a “closed” tip member. In other words, the tip member 26 may not include a distal opening that allows fluids and/or devices to pass therethrough.

An expandable balloon 32 may be coupled to the shaft 12. The balloon 32 may be similar in form and function to other balloons suitable for medical use. In some instances, the balloon 32 may be made from or otherwise include a compliant material or materials. Alternatively, the balloon 32 may be made from or otherwise include a non-compliant material or materials. The balloon 32 may be designed to be inflated to a relatively high pressure (e.g., suitable for expanding a lesion). Alternatively, the balloon 32 may be designed to be inflated to a relatively low pressure (e.g., suitable for occluding a vascular region).

The balloon 32 may include a proximal waist region 34 and a distal waist region 36. The waist regions 34, 36 may be coupled to the inner member 20, the outer member 24, or both. For example, the proximal waist region 34 may be coupled directly to the outer surface of the inner member 20. In at least some of these instances, it may be desirable to couple the proximal waist region 34 to a region of the inner member 20 that lacks slots 22. However, this may not be required. In other instances, the proximal waist region 34 may be coupled to the outer member 24. In at least some of these instances, the proximal waist region 34 may be coupled to a distal end region of the outer member 24. Accordingly, the outer member 24 may terminate at or near the proximal waist region 34. Similarly, the distal waist region 36 may be coupled directly to the outer surface of the inner member 20. In other instances, a sleeve 38 may be coupled to the outer surface of the inner member 20 and disposed between the distal waist region 36 and the inner member 20. In some of these and in other instances, a sleeve (not shown) may be disposed over the balloon 32 (e.g., over the proximal waist region 34, the distal waist region 36, or both), over the outer member 24 (if present adjacent to the proximal waist region 34), over the sleeve 38 (if present), and/or over the inner member 20.

As indicated herein, the inner member 20 may have a plurality of slots 22 formed therein. The slots 22 may be disposed along substantially the full length of the inner member 20 or along one or more portions of the inner member 20. For example, the inner member 20 may include a first region 40 having a first plurality of slots 22 formed therein and a second region 42 having a second plurality of slots 22 formed therein. A third region 44 of the inner member 20 may be disposed between the first region 40 and the second region 42. The third region may be free of slots 22. A fourth region 46 of the inner member 20 may be disposed adjacent to the second region 42. The fourth region 46 may be free of slots.

The first region 40 of the inner member 20 may be covered by the outer member 24 so that the outer member 24 substantially seals the first plurality of slots 22. Accordingly, inflation fluid may be passed through the inner member 20 in order to inflate the balloon 32. The second region 42 may be disposed underneath the balloon 32 and may be free of the outer member 24. This allows the inflation fluid to pass through the slots 22 along the second region 42 in order to inflate the balloon 32.

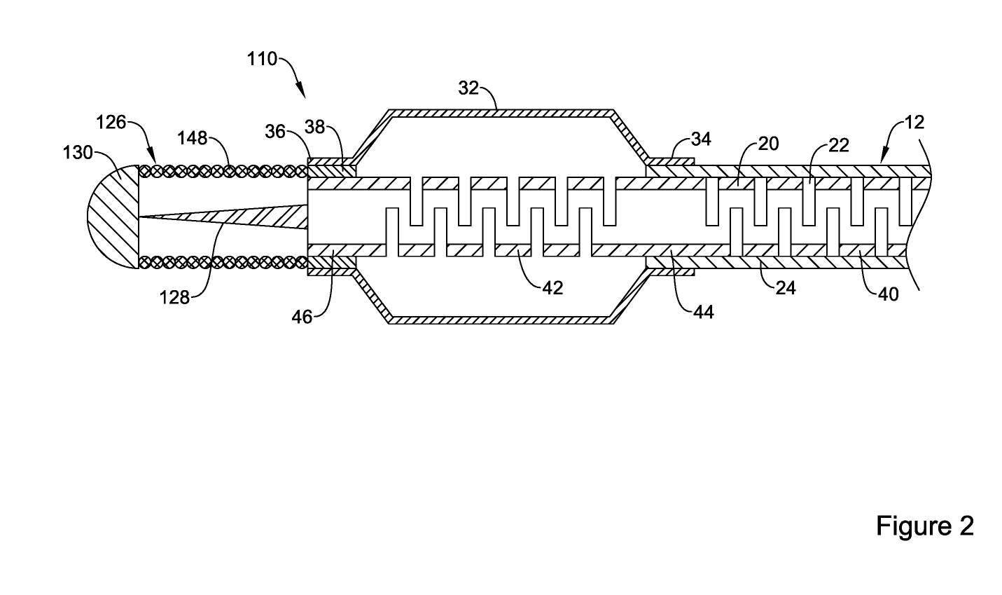

FIG. 2 illustrates another example medical device 110 that may be similar to other medical devices disclosed herein. In this example, the medical device 110 takes the form of a guidewire. In this example, the guidewire 110 may include a tip member 126 that takes the form of a spring tip. As such, the tip member 126 may include a shaping member 128, a coil or spring 148, and a tip 130. Other configurations are contemplated.

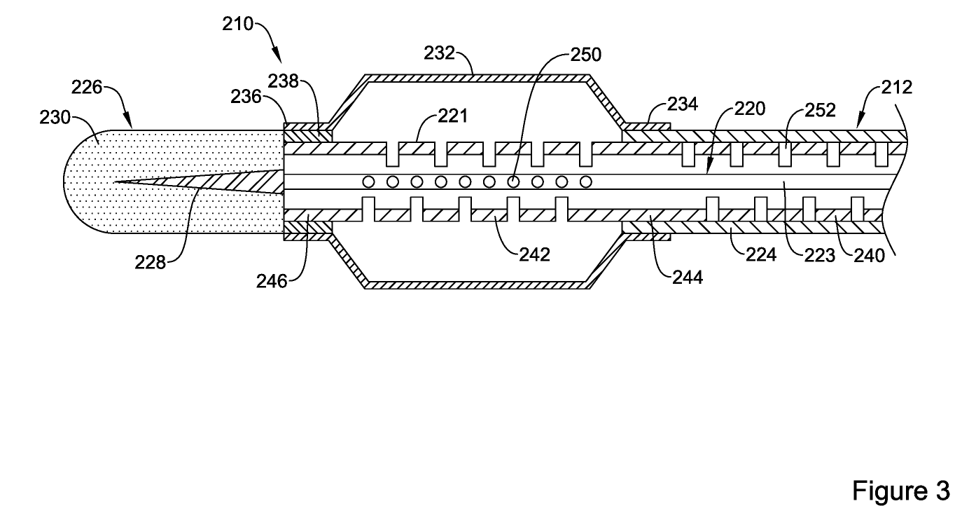

FIG. 3 illustrates another example medical device 210 that may be similar to other medical devices disclosed herein. In this example, the medical device 210 takes the form of a guidewire. The guidewire 210 may include an elongate shaft 212. The shaft 212 may include an inner tubular member 220 and an outer tubular member 221. It is noted that for clarity purposes, the inner tubular member 220 is not depicted in cross section. The outer tubular member 221 may be similar in form to the inner member 20 of the guidewire 10. For example, the outer tubular member 221 may have a plurality of slots 252 formed therein. In some instances, an outer sleeve or jacket 224 may be disposed along portions of or all of the outer tubular member 221. The inner tubular member 220 may be extend though the outer tubular member 221. In some instances, the inner tubular member 220 may be coaxial with the outer tubular member 221. Alternatively, the longitudinal axis of the inner tubular member 220 may be offset (e.g., radially offset) from the longitudinal axis of the outer tubular member 221.

In some instances, the inner tubular member 220 may define a lumen 223 (e.g., an inflation lumen). A plurality of openings 250 may be formed in the inner tubular member 220. Accordingly, a fluid (e.g., an inflation fluid) may be passed through the lumen 223 of the inner tubular member 220 and through the openings 250. In at least some instances, the fluid can also pass through the slots 252 of the outer tubular member 221 so that the fluid can inflate a balloon 232 coupled to the shaft 212. The openings 250 may have a variety of shapes, arrangements, and/or configurations. For example, the openings 250 may have a substantially round shape. Alternatively, the openings 250 may be oval or otherwise have a non-circular shape. Other shapes are contemplated.

Just like the balloon 32, the balloon 232 may include a proximal waist region 234 and a distal waist region 236. The waist regions 234, 236 may be coupled to the inner tubular member 220, the outer tubular member 221, or both. For example, the proximal waist region 234 may be coupled directly to the outer surface of the outer tubular member 221. In at least some of these instances, it may be desirable to couple the proximal waist region 234 to a region of the outer tubular member 221 that lacks slots 252. However, this may not be required. In other instances, the proximal waist region 234 may be coupled to the jacket 224 disposed along the outer tubular member 221. In at least some of these instances, the proximal waist region 234 may be coupled to a distal end region of the jacket 224. Accordingly, the jacket 224 may terminate at or near the proximal waist region 234. Similarly, the distal waist region 236 may be coupled directly to the outer surface of the outer tubular member 221. In other instances, a sleeve 238 may be coupled to the outer tubular member 221 and disposed between the distal waist region 236 and the outer tubular member 221.

As indicated herein, the outer tubular member 221 may have a plurality of slots 252 formed therein. The slots 252 may be disposed along substantially the full length of the outer tubular member 221 or along one or more portions of the outer tubular member 221. For example, the outer tubular member 221 may include a first region 240 having a first plurality of slots 252 formed therein and a second region 242 having a second plurality of slots 252 formed therein. A third region 244 of the outer tubular member 221 may be disposed between the first region 240 and the second region 242. The third region may be free of slots 252. A fourth region 246 of the outer tubular member 221 may be disposed adjacent to the second region 242. The fourth region 246 may be free of slots.

The first region 240 of the outer tubular member 221 may be covered by the jacket 224 so that the jacket 224 substantially seals the first plurality of slots 252. The second region 242 may be disposed underneath the balloon 232 and may be free of the jacket 224. This allows the inflation fluid to pass through the openings 250 and through the slots 252 along the second region 242 of the outer tubular member 221 in order to inflate the balloon 232.

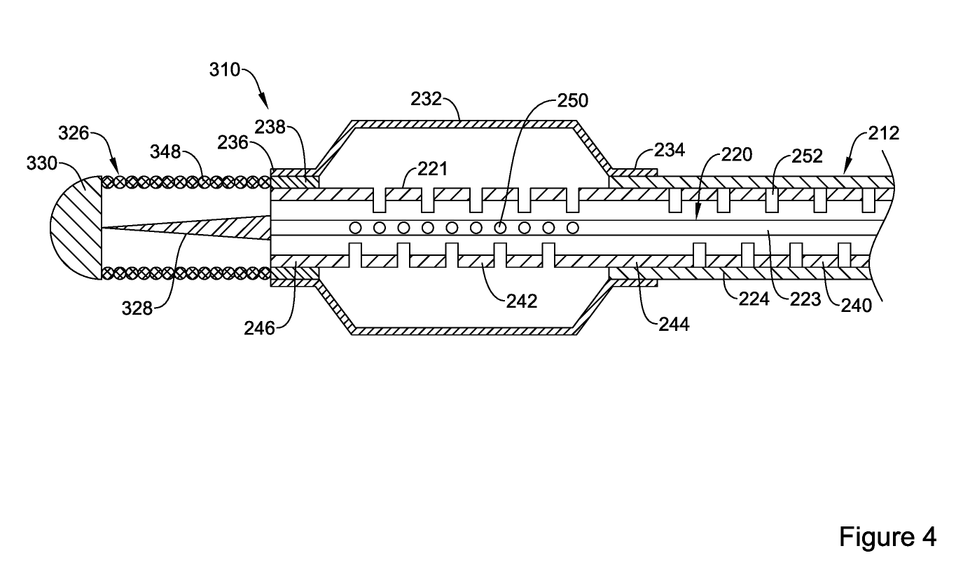

A tip member 226 may be coupled to the shaft 212 and extend distally therefrom. In some instances, the tip member 226 may include a shaping member or core member 228 and a jacket 230. In at least some instances, the tip member 226 is a “closed” tip member. In other words, the tip member 226 may not include a distal opening that allows fluids and/or devices to pass therethrough. Other tip members are contemplated. For example, FIG. 4 illustrates another example medical device 310 that may be similar to other medical devices disclosed herein. In this example, the medical device 310 may include a tip member 326 that takes the form of a spring tip. As such, the tip member 326 may include a shaping member 328, a coil or spring 348, and a tip 330.

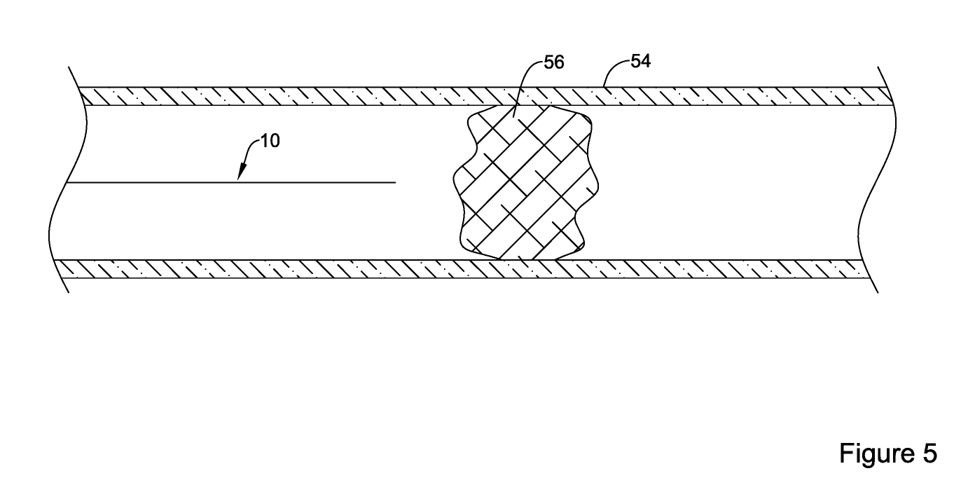

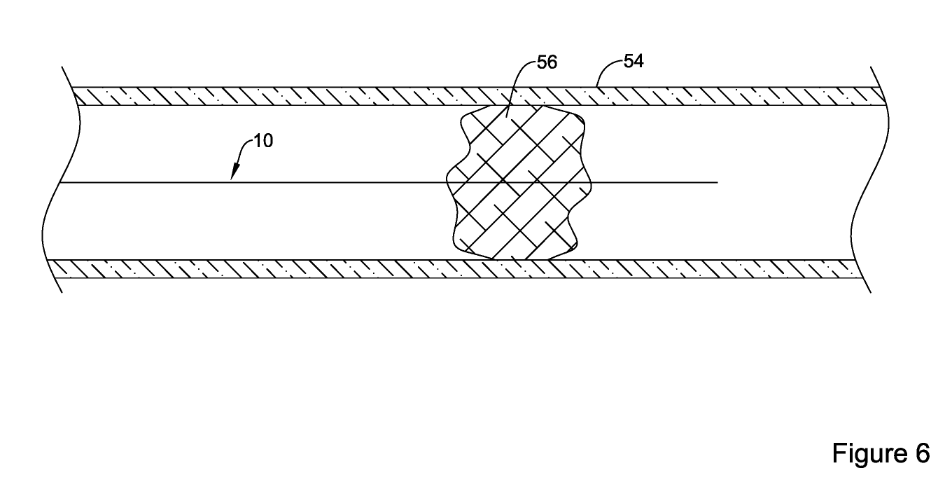

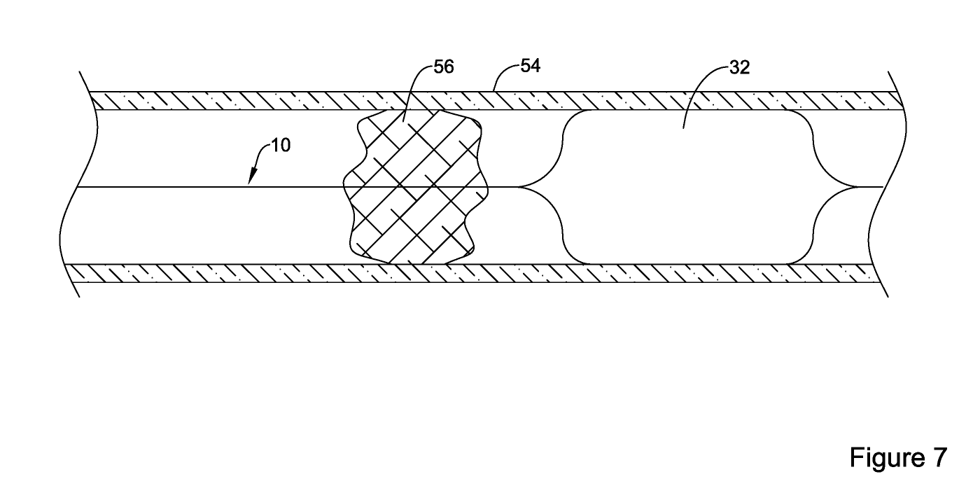

FIGS. 5-9 schematically illustrate an example process for using a medical device 10 (and/or any of the other medical devices and/or guidewires disclosed herein). For example, FIG. 5 illustrates the guidewire 10 disposed within a vascular region 54 adjacent to an occlusion or embolism 56. In this example, the vascular region 54 may be a neural blood vessel. However, other vascular regions are contemplated. In order to treat the embolism, the guidewire 10 may be advanced distally beyond or otherwise through the embolism 56 as shown in FIG. 6. With the guidewire 10 positioned distally beyond the embolism 56, the balloon 32 may be inflated as shown in FIG. 7. This may include passing inflation fluid through the inner member 20. The inflated balloon 32 may help to reduce distal migration of the embolism 56 during a medical procedure (e.g., distal protection), occlude flow in the vascular region 54, and/or the like.

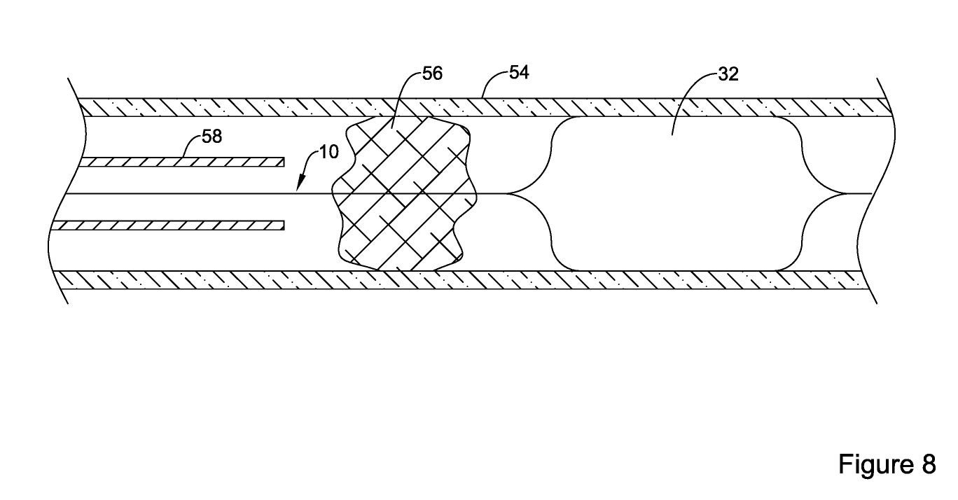

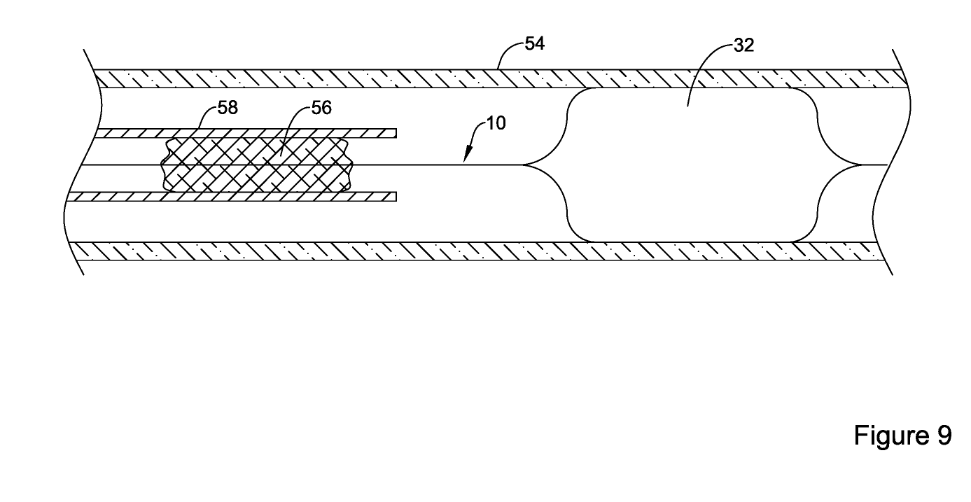

A medical device 58 may be advanced over the guidewire 10 to a position adjacent to the embolism 56 as shown in FIG. 8. The form of the medical device 58 may vary. For example, the medical device 58 may take the form of a thrombectomy catheter, aspiration catheter, microcatheter, wire, clot grabber, or the like. In this example, the medical device 58 may be used to aspirate the embolism 56 as shown in FIG. 9.

The use of the guidewire 10 (and/or other guidewires and/or medical devices disclosed herein) may be desirable for a number of reasons. For example, the guidewire 10 may be used as a primary wiring device and/or as a wire for the delivery of a second device thereover. The guidewire 10 may also be used to provide occlusion and/or distal protection during removal of an occlusion, clot, embolism, or the like. Because of the size and/or structural design of the guidewire 10, the guidewire 10 may be able to provide access to deeper/remote regions of the vasculature while be deliverable and maneuverable in a manner that minimizes trauma to the anatomy. Furthermore, the guidewire 10 may allow for the relatively quick removal of an occlusion, clot, embolism, or the like and, in at least some instances, allow for the removal in just one pass.

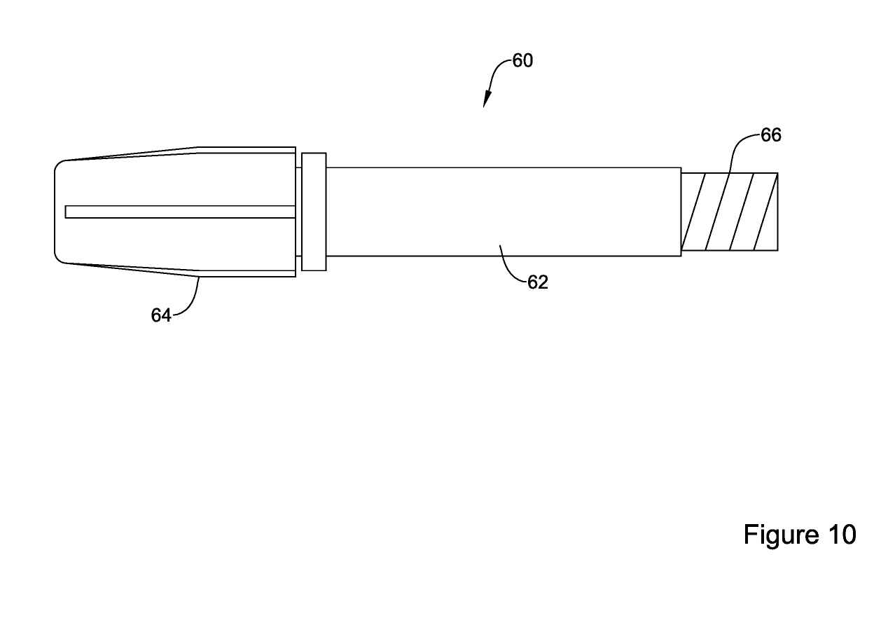

FIG. 10 is a side view of an accessory device 60 for use with the medical device 10 (and/or any of the other medical devices and/or guidewires disclosed herein). The accessory device 60 may take the form of a torque device that includes a body region 62. The accessory device 60 may include a first connector region 64 that may generally be designed to be coupled to the guidewire 10. Because the guidewire 10 may be designed to have fluid passed therethrough, the first connector region 64 may be designed to couple to the guidewire 10 while forming a fluid tight seal thereon. For example, the first connector region 64 may include a securing member (not shown) such a collet and a sealing member (not shown). The accessory device 60 may also include a second connector region 66. The second connector region 66 may be designed to allow another device such as a syringe, inflation device (e.g., insufflator), or the like to be coupled thereto. In some instances, the second connector region 66 may include a threaded and/or or luer connector. Other connectors are contemplated.

The materials that can be used for the various components of the medical device 10 (and/or any of the other medical devices and/or guidewires disclosed herein) and the various tubular members/shafts disclosed herein may include those commonly associated with medical devices. For simplicity purposes, the following discussion makes reference to inner member 20 and other components of the medical device 10. However, this is not intended to limit the devices and methods described herein, as the discussion may be applied to other similar tubular members and/or components of tubular members or devices disclosed herein.

Inner member 20 and/or other components of the medical device 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Various embodiments of arrangements and configurations of slots are also contemplated that may be used in addition to what is described above or may be used in alternate embodiments. For simplicity purposes, the following disclosure makes reference to the guidewire 10, the inner member 20, and the slots 22. However, it can be appreciated that these variations may also be utilized for other slots and/or openings disclosed herein. In some embodiments, at least some, if not all of the slots 22 are disposed at the same or a similar angle with respect to the longitudinal axis of the inner member 20. As shown, the slots 22 can be disposed at an angle that is perpendicular, or substantially perpendicular, and/or can be characterized as being disposed in a plane that is normal to the longitudinal axis of the inner member 20. However, in other embodiments, the slots 22 can be disposed at an angle that is not perpendicular, and/or can be characterized as being disposed in a plane that is not normal to the longitudinal axis of the inner member 20. Additionally, a group of one or more slots 22 may be disposed at different angles relative to another group of one or more slots 22. The distribution and/or configuration of the slots 22 can also include, to the extent applicable, any of those disclosed in U.S. Pat. Publication No. US 2004/0181174, the entire disclosure of which is herein incorporated by reference.

The slots 22 may be provided to enhance the flexibility of the inner member 20 while still allowing for suitable torque transmission characteristics. The slots 22 may be formed such that one or more rings and/or tube segments interconnected by one or more segments and/or beams that are formed in the inner member 20, and such tube segments and beams may include portions of the inner member 20 that remain after the slots 22 are formed in the body of the inner member 20. Such an interconnected structure may act to maintain a relatively high degree of torsional stiffness, while maintaining a desired level of lateral flexibility. In some embodiments, some adjacent the slots 22 can be formed such that they include portions that overlap with each other about the circumference of the inner member 20. In other embodiments, some adjacent the slots 22 can be disposed such that they do not necessarily overlap with each other, but are disposed in a pattern that provides the desired degree of lateral flexibility.

Additionally, the slots 22 can be arranged along the length of, or about the circumference of, the inner member 20 to achieve desired properties. For example, adjacent slots 22, or groups of slots 22, can be arranged in a symmetrical pattern, such as being disposed essentially equally on opposite sides about the circumference of the inner member 20, or can be rotated by an angle relative to each other about the axis of the inner member 20. Additionally, adjacent slots 22, or groups of slots 22, may be equally spaced along the length of the inner member 20, or can be arranged in an increasing or decreasing density pattern, or can be arranged in a non-symmetric or irregular pattern. Other characteristics, such as slot size, slot shape, and/or slot angle with respect to the longitudinal axis of the inner member 20, can also be varied along the length of the inner member 20 in order to vary the flexibility or other properties. In other embodiments, moreover, it is contemplated that the portions of the tubular member, such as a proximal section, or a distal section, or the entire the inner member 20, may not include any such slots 22.

As suggested herein, the slots 22 may be formed in groups of two, three, four, five, or more slots 22, which may be located at substantially the same location along the axis of the inner member 20. Alternatively, a single slot 22 may be disposed at some or all of these locations. Within the groups of slots 22, there may be included slots 22 that are equal in size (i.e., span the same circumferential distance around the inner member 20). In some of these as well as other embodiments, at least some slots 22 in a group are unequal in size (i.e., span a different circumferential distance around the inner member 20). Longitudinally adjacent groups of slots 22 may have the same or different configurations. For example, some embodiments of the inner member 20 include slots 22 that are equal in size in a first group and then unequally sized in an adjacent group. It can be appreciated that in groups that have two slots 22 that are equal in size and are symmetrically disposed around the tube circumference, the centroid of the pair of beams (e.g., the portion of the inner member 20 remaining after the slots 22 are formed therein) is coincident with the central axis of the inner member 20. Conversely, in groups that have two slots 22 that are unequal in size and whose centroids are directly opposed on the tube circumference, the centroid of the pair of beams can be offset from the central axis of the inner member 20. Some embodiments of the inner member 20 include only slot groups with centroids that are coincident with the central axis of the inner member 20, only slot groups with centroids that are offset from the central axis of the inner member 20, or slot groups with centroids that are coincident with the central axis of the inner member 20 in a first group and offset from the central axis of the inner member 20 in another group. The amount of offset may vary depending on the depth (or length) of the slots 22 and can include other suitable distances.

The slots 22 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the inner member 20 is formed by cutting and/or removing portions of the tube to form the slots 22. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference.

In at least some embodiments, the slots 22 may be formed in tubular member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow the inner member 20 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form the inner member 20 without being limited by a minimum cutting blade size. Consequently, the inner member 20 may be fabricated for use in neurological devices or other devices where a relatively small size may be desired.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A method for treating a vascular region, the method comprising:
advancing a guidewire through a neural blood vessel to a first position adjacent to an embolism, the guidewire comprising:
an elongate shaft having a distal end region, the elongate shaft including an inner member and an outer member,
wherein the inner member has a plurality of openings formed therein, and
an inflatable balloon coupled to the distal end region;
advancing the guidewire to a second position distal of the embolism;
inflating the inflatable balloon;
advancing a treatment catheter over the guidewire to a position adjacent to the embolism; and
aspirating the embolism.

2. The method of claim 1, wherein at least a portion of the guidewire has an outer diameter of 0.01 to 0.018 inches.

3. The method of claim 1, wherein the inner member includes a tubular member defining a lumen.

4. The method of claim 3, wherein the lumen is an inflation lumen, wherein the inflatable balloon is positioned about at least some of the plurality of openings, and wherein inflating the inflatable balloon includes passing inflation media through the inflation lumen and at least some of the plurality of openings.

5. The method of claim 1, wherein the outer member includes a polymeric sleeve.

6. The method of claim 1, wherein the outer member includes a tubular member with a region having a plurality of slots formed therein.

7. A guidewire for accessing and treating a region of the neural vasculature, the guidewire comprising:
an elongate shaft having a distal end region, the elongate shaft including an inner tubular member and an outer tubular member;
wherein the inner tubular member has a plurality of openings formed therein;
wherein the outer tubular member has a plurality of slots formed therein;
an occlusive balloon coupled to the distal end region of the elongate shaft and disposed about at least some of the plurality of openings;
wherein the inner tubular member defines a lumen in fluid communication with the occlusive balloon through the plurality of openings; and
a tip member coupled to the distal end region of the elongate shaft.

8. The guidewire of claim 7, wherein the plurality of slots include a first plurality of slots disposed along a first region of the outer tubular member, a second plurality of slots disposed along a second region of the outer tubular member, and a third region positioned between the first region and the second region that is free of slots.

9. The guidewire of claim 8, wherein the occlusive balloon includes a proximal waist and wherein the proximal waist is coupled to the second region.

10. The guidewire of claim 9, wherein the outer tubular member includes a fourth region disposed adjacent to the third region, the fourth region being free of slots.

11. The guidewire of claim 10, wherein the occlusive balloon includes a distal waist and wherein the distal waist is coupled to the fourth region.

12. The guidewire of claim 11, further comprising a sleeve disposed between the distal waist and the fourth region of the outer tubular member.

13. The guidewire of claim 7, wherein at least some of the plurality of openings in the inner tubular member are circular in shape.

14. The guidewire of claim 7, wherein at least some of the plurality of openings in the inner tubular member are non-circular in shape.

15. The guidewire of claim 7, wherein the tip member has a closed distal end.

16. The guidewire of claim 7, wherein the tip member includes a polymer jacket.

17. The guidewire of claim 7, wherein the tip member includes a coil.

18. The guidewire of claim 7, wherein the outer tubular member includes a distal portion, a proximal portion, and a connector coupling the distal portion to the proximal portion.

19. A guidewire for accessing and treating a relatively small vasculature region, the guidewire comprising:
an inner tubular member having a distal region with a plurality of openings formed therein;
an outer tubular member disposed about the inner tubular member, the outer tubular member having a first region having a first plurality of slots formed therein, a second region having a second plurality of slots formed therein, and a third region disposed between the first region and the second region, the third region being free of slots;
a balloon coupled to the third region of the outer tubular member;
wherein the inner tubular member defines a lumen in fluid communication with the balloon through the plurality of openings; and
a closed tip member extending distally from a distal end region of the outer tubular member.

20. The guidewire of claim 19, wherein the outer tubular member includes a fourth region free of slots and wherein a sleeve is disposed along the fourth region and positioned between the outer tubular member and the balloon.
